(19) United States
(12) Patent Application Publication
(10) Pub. No.: US 2019/0219506 A1
(43) Pub. Date: Jul. 18, 2019

(54) INSPECTION UNITS WITH ULTRAVIOLET RADIATION SOURCES OPERATING AT DIFFERENT WAVELENGTHS

(71) Applicant: GLOBALFOUNDRIES Inc., Grand Cayman (KY)

(72) Inventors: Tarek Mohamed Gould, Mechanicville, NY (US); Robert Carso, Ballston Spa, NY (US); Joshua Moore, Clifton Park, NY (US); Siddharth Bhat, Ballston Spa, NY (US); Joshua Kaplan, Clifton Park, NY (US)

(21) Appl. No.: 15/873,111

(22) Filed: Jan. 17, 2018

(51) Int. Cl. G01N 21/64

(52) U.S. Cl. CPC G01N 21/64; G01N 21/94; G01N 2201/061

(57) ABSTRACT

Inspection units and methods of inspecting objects for the presence of particle contamination. An object inside an inspection unit is exposed to ultraviolet radiation at a first wavelength and to ultraviolet radiation at a second wavelength that is less than the first wavelength. Fluorescence is emitted from particles on a surface of the object in response to the ultraviolet radiation at the first wavelength and in response to the ultraviolet radiation at the second wavelength, and is visible external of the inspection unit.

### BACKGROUND

The present invention relates to contamination control and, more specifically, to inspection units and methods of inspecting objects for the presence of particle contamination.

A cleanroom is a controlled environment in which products are manufactured, and in which the concentration of airborne particles is carefully controlled to specified limits. Particle contamination may be observed during pre-cleanroom and pre-preventative maintenance inspection of parts, tools, and materials. Contamination inspection may be useful, for example, in a gowning room before objects are transported from the gowning room into the cleanroom. Objects may be inspected to determine whether, for example, their state of cleanliness meets site standards before introducing those objects into the clean room.

Improved inspection units and methods of inspecting objects for the presence of particle contamination are needed.

### SUMMARY

In an embodiment of the invention, a method includes exposing an object inside an inspection unit to ultraviolet radiation at a first wavelength, and exposing the object inside the inspection unit to ultraviolet radiation at a second wavelength that is less than the first wavelength. The method further includes observing fluorescence emitted from particles on a surface of the object in response to the ultraviolet radiation at the first wavelength and in response to the ultraviolet radiation at the second wavelength.

In an embodiment of the invention, an apparatus includes a housing, a first source of ultraviolet radiation arranged inside the housing, and a second source of ultraviolet radiation arranged inside the housing. The first source is configured to emit the ultraviolet radiation at a first wavelength, and the second source is configured to emit the ultraviolet radiation at a second wavelength that is less than the first wavelength.

### BRIEF DESCRIPTION OF THE DRAWINGS

The accompanying drawings, which are incorporated in and constitute a part of this specification, illustrate various embodiments of the invention and, together with a general description of the invention given above and the detailed description of the embodiments given below, serve to explain the embodiments of the invention.

FIG. 1 is a diagrammatic view of an inspection unit in accordance with embodiments of the invention.

FIG. 2 is a diagrammatic view showing the interior of the inspection unit of FIG. 1.

### DETAILED DESCRIPTION

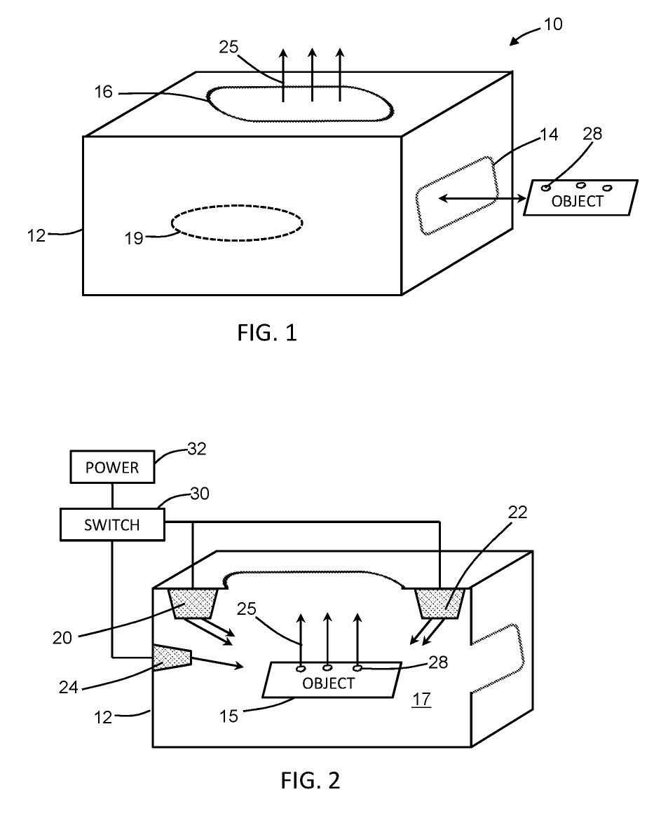

With reference to FIGS. 1, 2 and in accordance with embodiments of the invention, an inspection unit 10 includes a housing 12 with an access port 14 and an observation port 16. The access port 14 provides an opening through the wall of the housing 12 for the movement of an object 15 and may be sized according to an expected maximum object size. The object 15 may be introduced into the interior 17 of the housing 12 through the access port 14 and removed from the interior 17 of the housing 12 through the access port 14. For example, the object 15 may be a glove, a gloved hand, a tool, etc. to be used inside a cleanroom or other controlled environment. The object 15 may be inserted into the housing 12, held within the housing 12, manipulated inside the housing 12, and removed from the housing 12 by hand. Openings 19 may be provided in one or more of the sidewalls of the housing 12 in order to permit manipulation of the object 15 inside the interior 17 of the housing 12. The size of the openings 19 may be limited to reduce the ingress of extraneous ambient light from sources exterior to the housing 12. The observation port 16 may be an opening in the inspection unit 10 that is used for the purpose of observing the interior of the housing 12. The ports 14, 16 may be dimensioned and configured to limit the ingress of ambient light from sources exterior to the housing 12. For example, the observation port 16 may include a window that is fitted into an opening provided in the housing 12, and that may provide wavelength-sensitive light filtering.

Sources 20, 22, 24 of electromagnetic energy are positioned inside the housing 12 and are arranged within the housing 12 to irradiate the object 15 inside the inspection unit 10 with electromagnetic radiation of different wavelengths. The source 20 may be configured to generate and emit electromagnetic radiation in the ultraviolet energy band of the electromagnetic spectrum. Similarly, the source 22 may be configured to generate and emit electromagnetic radiation in the ultraviolet energy band of the electromagnetic spectrum, but at a different wavelength (i.e., energy) than the source 20.

In an embodiment, the source 20 may generate and emit electromagnetic radiation at a wavelength that is less than (i.e., shorter than) the wavelength of the electromagnetic radiation generated and emitted by the source 22. In an embodiment, the source 20 may generate and emit electromagnetic radiation at a wavelength within the UV-C band of the electromagnetic spectrum, and the source 22 may generate and emit electromagnetic radiation at a wavelength within the UV-A band of the electromagnetic spectrum. In an embodiment, the source 20 may generate and emit electromagnetic radiation at a wavelength within a range of 200 nanometers to 280 nanometers, and the source 22 may generate and emit electromagnetic radiation at a wavelength within a range of 315 nanometers to 390 nanometers. In connection with these different embodiments, each of the sources 20 and 22 may be monochromatic and therefore include electromagnetic radiation of either a single wavelength or a very small band or range of wavelengths. In an embodiment, the source 20 may generate and emit electromagnetic radiation at a wavelength of 254 nanometers or a narrow range of wavelengths centered about 254 nanometers, and the source 22 may generate and emit electromagnetic radiation at a wavelength of 365 nanometers or a narrow range of wavelengths centered about 365 nanometers.

The electromagnetic radiation of the source 20 and the source 22 may cause particles 28 present on the surface of the object 15 inserted into the interior of the housing 12 to fluoresce. The particles 28 on the object 15 may absorb the electromagnetic radiation from one or both of the sources 20, 22, and respond by fluorescing to emit florescence 25 (i.e., light) with a longer wavelength (i.e., lower energy) than the absorbed ultraviolet radiation from either of the sources 20, 22. The wavelength of the florescence 25 emitted from the particles 28 on the object 15 may fall within the visible portion of the electromagnetic spectrum to which the human eye can respond, and may be observable externally to the housing 12 of the inspection unit 10 as light emission through the observation port 16 in the housing 12.

The wavelength selected for the source 20 and the wavelength selected for the source 22 may each cause different intensities or levels of fluorescence 25 from irradiated particles 28 of different types, compositions, etc. on the inserted object 15. For example, particles 28 composed of an inorganic material (e.g., one or more mineral compounds and/or elements) may emit fluorescence 25 of a given intensity when irradiated by the electromagnetic radiation from the source 20. For example, particles 28 composed of an organic material (e.g., polymeric fibers) may emit fluorescence 25 of a different given intensity when irradiated by the electromagnetic radiation from the source 22. The particles 28 have a definable shape and/or mass, and a particle size that may be represented by a maximum linear dimension or diameter. A fiber is a type of particle that may have, for example, a length-to-width ratio exceeding 10:1. Through the use of different wavelengths of ultraviolet radiation, particles 28 of different compositions may be caused to fluoresce with observable intensities of fluorescence 25 within the same confined space (i.e., the confined space inside the housing 12 of the inspection unit 10).

Each of the sources 20 and 22 may include one or more bulbs, lamps, light-emitting diodes (LEDs), etc. selected to provide a targeted intensity of electromagnetic radiation inside the housing 12. In an embodiment, each of the sources 20 and 22 may include three to four bulbs, lamps, or LEDs. The interior surfaces of the housing 12 may be coated with a reflective material, such as a magnesium-based compound, to effectively increase the intensity through reflection of the electromagnetic radiation.

The electromagnetic radiation from one or both of the sources 20, 22 may also sterilize and sanitize the irradiated object 15 inside the inspection unit 10. To that end, the electromagnetic radiation, particularly that emitted by the source 20 at a shorter wavelength, may kill microorganisms (e.g., microbes) resident on the surfaces of the irradiated object 15 so that the object 15 is disinfected or, at the least, has a smaller density of microorganisms following irradiation.

The source 24 may be configured to generate and emit electromagnetic radiation in the visible band of the electromagnetic spectrum. In an embodiment, the source 24 may generate and emit electromagnetic radiation at one or more wavelengths that are greater than (i.e., longer than) the wavelength generated and emitted by the source 20 and/or that are greater than the wavelength generated and emitted by the source 22. In an embodiment, the source 24 may generate and emit electromagnetic radiation at a wavelength or over a range of wavelengths within a range of 390 nanometers to 700 nanometers. In an embodiment, the source 24 may emit white light that includes components at a combination of wavelengths within the visible spectrum. The luminous flux emitted from the source 24 may be greater than or equal to 200 lumens.

The source 24 may be mounted and oriented such that its electromagnetic radiation impinges the object 15 inserted into the housing 12 at a small or shallow incidence angle relative to horizontal. In an embodiment, the incidence angle may be less than or equal to 30° from horizontal. In an embodiment, the incidence angle may be less than or equal to 15° from horizontal. The shallow angle of incidence will illuminate particles 28 on the inserted object 15 by, for example, reflection, and the reflected light is observable externally through the observation port 16. The particles 28 that are illuminated and visualized may only have weak fluorescence or no fluorescence such that the electromagnetic radiation from the source 24 provides a primary visualization mechanism.

The sources 20, 22, 24 may be switched on and off using a switch 30, which is preferably located externally to the housing 12 of the inspection unit 10, connected between a power supply 32 and the sources 20, 22, 24. The external location of the switch 30 assists in limited cross-contamination between successively inserted objects. The switch 30 may also be used to distribute electric power between the sources 20, 22, 24. In various embodiments, the sources 20, 22, 24 may be powered sequentially or simultaneously by switched electric power from the power supply 32, and/or may be powered individually or in combination. In an embodiment, the switch 30 may be used to power the sources 20, 22, 24 sequentially and individually such that only one of the sources 20, 22, 24 is powered and illuminating the object 15 at any one time.

The inspection unit may be used to visually detect particles, such as macro-particles, contaminating the exterior surfaces of objects, such as parts, tools and materials, during pre-cleanroom and pre-preventative maintenance inspection by exposure to visible and ultraviolet radiation. Macro-particles with particle sizes greater than or equal to 0.5 microns may be most effectively detected. The inspection unit may be fixed in position or may be integrated with a mobile cart that can be placed at selected module or tool locations in a fabrication facility for the purpose of controlling cross-contamination during preventative maintenance. The inspection unit may be used to educate cleanroom personnel in cleanliness of, for example, gloves on gloved hands inserted into the housing. The inspection unit may be used to test the effectiveness of different cleaning (e.g., wiping) methods for removing particle contamination, and is intended for qualitative measurements, as opposed to quantitative measurements. The inspection unit is best suited for diagnostic purposes relating to indicating a non-quantitative number or density of particles on an object. The objects placed into the inspection unit may include, but are not limited to, gloves, phones, tablet computers, glasses, notebooks, tools, machine parts, and other types of objects entering a cleanroom.

References herein to terms such as “vertical”, “horizontal”, “lateral”, etc. are made by way of example, and not by way of limitation, to establish a frame of reference. Terms such as “horizontal” and “lateral” refer to a direction in a plane parallel to a top surface of a semiconductor substrate, regardless of its actual three-dimensional spatial orientation. Terms such as “vertical” and “normal” refer to a direction perpendicular to the “horizontal” and “lateral” direction. Terms such as “above” and “below” indicate positioning of elements or structures relative to each other and/or to the top surface of the semiconductor substrate as opposed to relative elevation.

A feature “connected” or “coupled” to or with another element may be directly connected or coupled to the other element or, instead, one or more intervening elements may be present. A feature may be “directly connected” or “directly coupled” to another element if intervening elements are absent. A feature may be “indirectly connected” or “indirectly coupled” to another element if at least one intervening element is present.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

1. An apparatus comprising:
a housing including a plurality of interior surfaces;
a first source of ultraviolet radiation arranged inside the housing, the first source configured to emit the ultraviolet radiation at a first wavelength towards a surface of an object disposed within the housing; and
a second source of ultraviolet radiation arranged inside the housing, the second source configured to emit the ultraviolet radiation towards the surface of the object at a second wavelength that is less than the first wavelength; and
a reflective material arranged on the interior surfaces of the housing to reflect the ultraviolet radiation at the first wavelength emitted by the first source or the ultraviolet radiation at the second wavelength emitted by the second source toward the surface of the object.

2. The apparatus of claim 1 wherein the first wavelength of the ultraviolet radiation emitted by the first source is within the ultraviolet C band of the electromagnetic spectrum, and the second wavelength of the ultraviolet radiation emitted by the second source is within the ultraviolet A band of the electromagnetic spectrum.

3. The apparatus of claim 1 wherein the first wavelength of the ultraviolet radiation emitted by the first source is within a range of 200 nanometers to 280 nanometers, and the second wavelength of the ultraviolet radiation emitted by the second source is within a range of 315 nanometers to 390 nanometers.

4. The apparatus of claim 1 wherein the first wavelength of the ultraviolet radiation emitted by the first source is 254 nanometers, and the second wavelength of the ultraviolet radiation emitted by the second source is 365 nanometers.

5. The apparatus of claim 1 further comprising:
a third source of visible radiation inside the housing, the third source configured to emit the visible radiation at one or more wavelengths in the visible range of the electromagnetic spectrum, and the third source is configured to emit the visible radiation with a luminous flux that is greater than or equal to 200 lumens.

6. (canceled)

7. The apparatus of claim 5 wherein the third source is configured to project the visible radiation with an incidence angle of less than or equal to 30° relative to the surface of the object inside the housing.

8. The apparatus of claim 1 further comprising:
a power supply; and
a switch coupling the power supply with the first source and the second source,
wherein the switch is arranged external to the housing.

9. The apparatus of claim 8 wherein the switch is configured to independently power the first source and the second source.

10. A method comprising:
exposing a surface of an object inside an inspection unit to ultraviolet radiation at a first wavelength;
reflecting the ultraviolet radiation at the first wavelength emitted by the first source toward the surface of the object;
exposing the surface of the object inside the inspection unit to ultraviolet radiation at a second wavelength that is less than the first wavelength;
reflecting the ultraviolet radiation at the second wavelength emitted by the second source toward the surface of the object; and
observing fluorescence emitted from particles on the surface of the object in response to the ultraviolet radiation at the first wavelength and in response to the ultraviolet radiation at the second wavelength.

11. The method of claim 10 wherein the object is exposed to the ultraviolet radiation at the first wavelength before the object is exposed to the ultraviolet radiation at the second wavelength.

12. The method of claim 10 further comprising:
exposing the object to visible radiation at one or more wavelengths in the visible range of the electromagnetic spectrum; and
observing the visible radiation reflected from the particles on the surface of the object.

13. The method of claim 12 wherein the visible radiation is emitted with a luminous flux that is greater than or equal to 200 lumens.

14. The method of claim 12 wherein the visible radiation is incident with an incidence angle of less than or equal to 30° relative to the surface of the object inside the inspection unit.

15. The method of claim 12 wherein the object is exposed to the ultraviolet radiation at the first wavelength before the object is exposed to the ultraviolet radiation at the second wavelength, and the object is exposed to the visible radiation after the object is exposed to the ultraviolet radiation at the second wavelength.

16. The method of claim 10 wherein the ultraviolet radiation at the first wavelength is emitted from a first source and the ultraviolet radiation at the second wavelength is emitted from a second source, the inspection unit includes a housing with an interior in which the first source and the second source are arranged, and further comprising:
inserting the object into the interior of the housing through an access port in the housing: and
observing the fluorescence emitted from the particles through an observation port in the housing.

17. The method of claim 16 wherein the inspection unit includes a switch located external to the housing, and further comprising:
after the object is inserted, operating the switch to sequentially operate the first source and the second source.

18. The method of claim 10 wherein the first wavelength of the ultraviolet radiation emitted by the first source is within the Ultraviolet C band of the electromagnetic spectrum, and the second wavelength of the ultraviolet radiation emitted by the second source is within the Ultraviolet A band of the electromagnetic spectrum.

19. The method of claim 10 wherein the first wavelength of the ultraviolet radiation emitted by the first source is within a range of 200 nanometers to 280 nanometers, and the second wavelength of the ultraviolet radiation emitted by the second source is within a range of 315 nanometers to 390 nanometers.

20. The method of claim 10 wherein the first wavelength of the ultraviolet radiation emitted by the first source is 254 nanometers, and the second wavelength of the ultraviolet radiation emitted by the second source is 365 nanometers.

21. The apparatus of claim 1 wherein fluorescence is emitted from particles on the surface of the object in response to the ultraviolet radiation at the first wavelength and in response to the ultraviolet radiation at the second wavelength, the housing includes an access port dimensioned for inserting the object into the housing, the housing includes an observation port configured for observing the fluorescence emitted from the particles, and the observation port includes a wavelength-sensitive light-filtering window fitted into an opening provided in the housing.
